(19) United States
(12) Patent Application Publication
(10) Pub. No.: US 2022/0331341 A1
(43) Pub. Date: Oct. 20, 2022

(54) TREATMENT COMPRISING THE USE OF FXR AGONISTS

(71) Applicant: Novartis AG, Basel (CH)

(72) Inventors: Dominique BREES, Arlesheim (CH); Patricia LOPEZ, Basel (CH); Iwona KSIAZEK, Basel (CH)

(21) Appl. No.: 17/762,666

(22) PCT Filed: Sep. 29, 2020
(86) PCT No.: PCT/IB2020/059110

§ 371 Date: Mar. 22, 2022

Related U.S. Application Data
(60) Provisional application No. 62/908,222, filed on Sep. 30, 2019.

(51) Int. Cl. A61K 31/575; A61K 31/46

(52) U.S. Cl. CPC A61K 31/575; A61K 31/46

(57) ABSTRACT

The invention provides, FXR agonists for the treatment of a condition or a disease associated with mitochondrial dysfunction, e.g. a mitochondrial disease, in a subject in need thereof.

### FIELD OF THE INVENTION

The present invention relates to methods of treating, preventing, or ameliorating conditions or diseases associated with mitochondrial dysfunction (e.g. for which mitochondrial dysfunction is major mechanism), comprising administering to a subject in need thereof a therapeutically effective amount of a FXR agonist. Furthermore, the invention is directed to the use of a farnesoid X receptor agonist (FXR agonist), such as tropifexor, for treating or preventing such diseases or disorders.

### BACKGROUND OF THE INVENTION

Nonalcoholic fatty liver disease (NAFLD) is the most common cause of chronic liver disease in the Western world. Non-alcoholic steatohepatitis (NASH), a more serious form of NAFLD is a worldwide problem with growing prevalence over the last few decades. Mitochondrial oxidative dysfunction is central to development and progression of NASH. Progression of NAFL to nonalcoholic fatty liver disease (NAFLD), starting with nonalcoholic steatohepatitis (NASH), involves intrahepatic inflammation. This process is associated with dysmorphologies, crystalline inclusions and increased amount of mutations in mitochondrial DNA.

More generally, optimizing mitochondrial health is advantageous for treating any disease. Primary dysfunction of mitochondria leads to progressive muscular and neurological degeneration. Generalized loss of mitochondria including liver mitochondria can result in hyperlipidemia, hypertension, and insulin resistance progression to Type 2 diabetes. Diseases or conditions associated with mitochondrial dysfunction, or mitochondrial diseases are a group of metabolic disorders, ranging from mild to severe, some can be fatal.

Mitochondrial hepatopathies includes primary disorders, in which the mitochondrial defect is the primary cause of the liver disorder, and secondary disorders, in which a secondary insult to mitochondria is caused by either a genetic defect that affects nonmitochondrial proteins or by an acquired (exogenous) injury to mitochondria (Sokol R J, Treem W R. Mitochondria and childhood liver diseases. J Pediatr GastroenterolNutr 1999; 28:4-16). Treatment of acute liver failure and progressive liver disease in the mitochondrial hepatopathies include medical therapies such as vitamins, cofactors, respiratory substrates, or antioxidant compounds, and liver transplantation.

The medical therapies used for treatment of mitochondrial disease have not however proven to be effective.

The FXR agonist tropifexor (see Tully et al, J Med Chem 2017; 60:9960-9973) is currently tested in nonalcoholic steatohepatitis patients with fibrosis (see NCT02855164 study). The compound was disclosed for the first time in WO 2012/087519 (Example 1, compound 1-IB of the table on page 125) and it is known under the name LJN452, or its international non-proprietary name tropifexor.

Treatment options for conditions or diseases for which mitochondrial dysfunction is major mechanism are currently limited and there remains a need for prophylactic and therapeutic approaches for the treatment of these conditions associated with mitochondrial dysfunction and toxicity. Thus, there is a need for treatments that stimulate mitochondrial function in response to increased metabolic demand and induce mitochondrial replication in response to agents or conditions that cause depletion of mitochondria in one or more tissues.

### SUMMARY OF THE INVENTION

The present invention relates, in part, to the finding that FXR activation by an FXR agonist, for example tropifexor, can restore mitochondrial dysfunction. The present invention also relates, in part, to the finding that FXR agonist is able to restore the hepatic mitochondrial dysfunction.

Accordingly, the present invention is directed to methods of treating, preventing, or ameliorating conditions associated with mitochondrial dysfunction, e.g mitochondrial diseases comprising administering to a subject in need thereof a therapeutically effective amount of a FXR agonist. Such conditions can be for example conditions mediated by farnesoid X receptors (FXRs). Furthermore, the invention is directed to the use of a farnesoid X receptor agonist (FXR agonist), such as tropifexor, for treating or preventing such diseases or disorders.

The invention also relates to methods of treating, preventing, or ameliorating conditions associated with mitochondrial dysfunction, in particular liver diseases or intestinal diseases, comprising administering to a subject in need thereof a therapeutically effective amount of a FXR agonist, wherein the administration of the FXR agonist to said subject is restoring the mitochondrial dysfunction, for example restoring mitochondrial dysfunction in hepatic cells.

The invention relates to methods of treating, preventing, or ameliorating conditions associated with mitochondrial dysfunction, in particular liver diseases or intestinal diseases, comprising administering to a subject in need thereof a therapeutically effective amount of a FXR agonist of formula

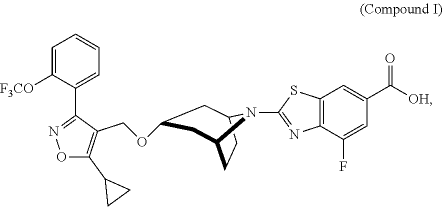

i.e. 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid), in free form, or a pharmaceutically acceptable salt thereof or an amino acid conjugate thereof, also known as tropifexor, wherein the administration of the FXR agonist to said subject is restoring the hepatic mitochondrial dysfunction.

The invention relates to methods of treating, preventing, or ameliorating conditions associated with mitochondrial dysfunction, for example liver injury, kidney ischemia reperfusion (I/R) injury, comprising administering to a subject in need thereof a therapeutically effective amount of a FXR agonist of formula

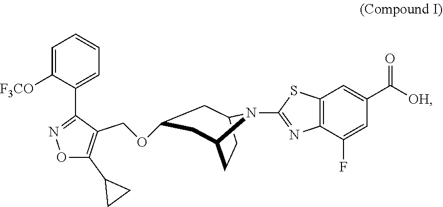

i.e. 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid), in free form, or a pharmaceutically acceptable salt thereof or an amino acid conjugate thereof, also known as tropifexor, and optionally wherein the administration of the FXR agonist to said subject is administered in the evening.

The invention further relates to methods of treating, preventing, or ameliorating mitochondrial hepatopathies comprising administering to a subject in need thereof a therapeutically effective amount of a FXR agonist of formula

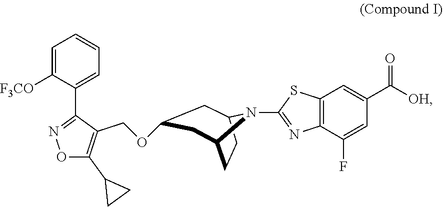

i.e. 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid), in free form, or a pharmaceutically acceptable salt thereof or an amino acid conjugate thereof, and optionally, wherein the FXR agonist is administered to said subject in the evening.

The invention provides new treatment regimens containing at least one FXR agonist, such as for example tropifexor, wherein the administration of the FXR agonist is occurring in the morning or in the evening. The treatment regimens wherein the administration of the FXR agonist is occurring in the evening, according to the present invention, offer the benefit of a high therapeutic efficacy while having low incidence of side effects, such as itching and/or lipid abnormalities (e.g. increased LDL cholesterol), which are, observed while using conventional treatment regimen. These treatment regimens further provide subjects with a convenient once daily dosing, thus supporting patient compliance.

Further features and advantages of the invention will become apparent from the following detailed description of the invention.

### BRIEF DESCRIPTION OF DRAWINGS

FIG. 1 shows that tropifexor decreases oxidative stress and restores antioxidant defenses.

FIG. 2 shows that tropifexor treatment promotes healthy mitochondrial function.

FIG. 3 provide evidence of restoration of mitochondrial function by tropifexor in NASH mice: tropifexor attenuates cleaved-caspase 3 levels, expression of anti-apoptotic genes are increased in tropifexor-treated groups.

FIG. 4 shows that FXR agonism by tropifexor abrogates hepatic mitochondrial dysfunction in NASH mice by increasing the expression of mitochondrial proteins, improving respiratory chain function, TCA cycle and ATP production.

### DETAILED DESCRIPTION OF THE INVENTION

It has been found that administering a FXR agonist to a subject in need thereof, for example tropifexor, can restore mitochondrial dysfunction. It has also been found that an FXR agonist is able to restore the hepatic mitochondrial dysfunction. The FXR agonist according to the invention can therefore be used to treat or prevent conditions for which mitochondrial dysfunction is a major mechanism.

The term ‘mitochondrial dysfunction’ has been frequently applied to describe either alteration in mitochondrial content, mitochondrial activity and/or submaximal ADP-stimulated oxidative phosphorylation under various physiological conditions. More generally, optimizing mitochondrial health is advantageous for treating any disease. Generalized loss of mitochondria including liver mitochondria can result in hyperlipidemia, hypertension, and insulin resistance progression to Type 2 diabetes. Liver mitochondria are injured by fructose uptake. Fructose, uric acid, and other agents injurious to liver mitochondria can cause accumulation of intracellular lipids, particularly triglycerides that contribute to the syndrome of hepatic steatosis, and increased synthesis and export of triglycerides that contributes to systemic hyperlipidemia, and ultimately obesity and insulin resistance. Progression of NAFL to nonalcoholic fatty liver disease (NAFLD), starting with nonalcoholic steatohepatitis (NASH), involves intrahepatic inflammation. This process is associated with dysmorphologies, crystalline inclusions and increased amount of mutations in mitochondrial DNA.

Ischemic and ischemia/reperfusion injury are accompanied by decreases in mitochondrial function and number, leading to apoptotic cell death, necrosis, and functional organ deterioration in ischemic conditions such as myocardial infarction and stroke. Despite considerable advances in the diagnosis and treatment of such conditions, there remains a need for prophylactic and therapeutic approaches for the treatment of these conditions. Acute kidney injury (AKI) is a common complication in patients undergoing major cardiac surgery, those receiving nephrotoxic drugs and those experiencing hemorrhage, dehydration or septic shock. Both inflammation and oxidative stress are critical for tissue destruction during kidney ischemia reperfusion (I/R) injury.

Mitochondrial hepatopathies refers to a plurality of disease, as disclosed in Sokol R J, Treem W R. Mitochondria and childhood liver diseases. J Pediatr GastroenterolNutr 1999; 28:4-16., and include (i) primary mitochondrial hepatopathies in which the mitochondrial defect is the primary cause of the liver disorder, and (ii) secondary mitochondrial hepatopathies, in which a secondary insult to mitochondria is caused by either a genetic defect that affects nonmitochondrial proteins or by an acquired (exogenous) injury to mitochondria.

Primary mitochondrial hepatopathies include, but are not limited to:

a) Electron transport (respiratory chain) defects: Neonatal liver failure (Complex I deficiency, Complex IV deficiency (SCO1 mutations), Complex III deficiency (BCS1L mutations), Multiple complex deficiencies), Mitochondrial DNA depletion syndrome (DGUOK, MPV17, and POLG mutations), Delayed onset liver failure (Alpers-Huttenlocher syndrome (POLG mutations)), Pearson marrow-pancreas syndrome (mitochondrial DNA deletion), Mitochondrial neurogastrointestinal encephalomyopathy (TP mutations), Chronic diarrhea (villus atrophy) with hepatic involvement (complex III deficiency), Navajo neurohepatopathy (mitochondrial DNA depletion; MPV17 mutations), Electron transfer flavoprotein (ETF) and ETF-dehydrogenase deficiencies;

b) Fatty acid oxidation and transport defects: Long-chain hydroxyacyl coenzyme A dehydrogenase deficiency, Acute fatty liver of pregnancy (long-chain hydroxyacyl coenzyme Adehydrogenase enzyme mutations), Carnitine palmitoyl transferase I and II deficiencies, Carnitine-acylcarnitine translocase deficiency, Fatty acid transport defects;

c) Disorders of mitochondrial translation process;

d) Urea cycle enzyme deficiencies;

e) Phosphoenolpyruvate carboxykinase deficiency (mitochondrial).

Secondary mitochondrial hepatopathies include, but are not limited to: Reye syndrome, Wilson's disease, valproic acid hepatotocixity, and the effects of nucleoside reverse transcriptase inhibitors.

Mitochondrial dysfunction is involved in the onset of nonalcoholic fatty liver disease (NAFLD) and contributes to the progression from NAFLD to nonalcoholic steatohepatitis (NASH). Targeting subcellular organelles that are fundamental in the pathogenic process of conditions, such as mitochondria, new therapeutic applications which may act both on mitochondrial function and energy supply and on regulators of lipid metabolism, such as FXR agonists disclosed herein, shall provide effective treatment of diseases or conditions for which mitochondrial dysfunction is a major mechanism.

Various (enumerated) embodiments of the present invention are described herein. It will be recognized that features specified in each embodiment may be combined with other specified features to provide further embodiments of the present disclosure.

### Embodiments (a)

1a: A FXR agonist for use in the treatment or in the prevention of a condition associated with mitochondrial dysfunction, wherein the FXR agonist is administered to a subject in need thereof at a therapeutically effective dose.

2a. A FXR agonist for use in the treatment or in the prevention of a condition associated with mitochondrial dysfunction and which condition is mediated by farnesoid X receptors (FXRs), wherein the FXR agonist is administered to a subject in need thereof at a therapeutically effective dose.

3a. A FXR agonist for use according to embodiments 1a or 2a, wherein the condition is a mitochondrial disease; e.g. any condition selected from the group consisting of: neurodegenerative diseases; cardiovascular diseases; diabetes and metabolic syndrome; autoimmune diseases; neurobehavioral and psychiatric diseases; gastrointestinal disorders; fatiguing illnesses; musculoskeletal diseases; cancer; chronic infections; and kidney injury and diseases; optionally wherein the condition is any condition selected from the group consisting of: acute kidney injury, hyperlipidemia, hypertension, insulin resistance and Type 2 diabetes.

4a. A FXR agonist for use in the treatment or in the prevention of a mitochondrial hepatopathy, wherein the FXR agonist is administered at a therapeutically effective dose.

5a. The FXR agonist for use according to Embodiment 4a, wherein the mitochondrial hepatopathy is a primary mitochondrial hepatopathy

6a. The FXR agonist for use according to Embodiment 5a, wherein the primary mitochondrial hepatopathy is selected from the group consisting of:

a) Electron transport (respiratory chain) defects: Neonatal liver failure (Complex I deficiency, Complex IV deficiency (SCO1 mutations), Complex III deficiency (BCS1L mutations), Multiple complex deficiencies), Mitochondrial DNA depletion syndrome (DGUOK, MPV17, and POLG mutations), Delayed onset liver failure (Alpers-Huttenlocher syndrome (POLG mutations)), Pearson marrow-pancreas syndrome (mitochondrial DNA deletion), Mitochondrial neurogastrointestinal encephalomyopathy (TP mutations), Chronic diarrhea (villus atrophy) with hepatic involvement (complex III deficiency), Navajo neurohepatopathy (mitochondrial DNA depletion; MPV17 mutations), Electron transfer flavoprotein (ETF) and ETF-dehydrogenase deficiencies;

b) Fatty acid oxidation and transport defects: Long-chain hydroxyacyl coenzyme A dehydrogenase deficiency, Acute fatty liver of pregnancy (long-chain hydroxyacyl coenzyme Adehydrogenase enzyme mutations), Carnitine palmitoyl transferase I and II deficiencies, Carnitine-acylcarnitine translocase deficiency, Fatty acid transport defects;

c) Disorders of mitochondrial translation process;

d) Urea cycle enzyme deficiencies;

e) Phosphoenolpyruvate carboxykinase deficiency (mitochondrial).

7a. The FXR agonist for use according to Embodiment 4a, wherein the mitochondrial hepatopathy is a secondary mitochondrial hepatopathy.

8a. The FXR agonist for use according to Embodiment 7a, wherein the secondary mitochondrial hepatopathy is selected from the group consisting of: Reye syndrome, Wilson's disease, valproic acid hepatotocixity, and the effects of nucleoside reverse transcriptase inhibitors.

9a: The FXR agonist for use according to any one of the preceding Embodiments, wherein the FXR agonist is administered to said subject once daily at a therapeutically effective dose, and wherein the FXR agonist is administered in the evening.

10a. The FXR agonist for use according to any one of the preceding Embodiments, wherein the FXR agonist is selected from tropifexor, obeticholic acid, nidufexor, cilofexor, TERN-101, EDP-305, PXL007, AGN242266 and MET409.

11a. The FXR agonist for use according to any one of the preceding Embodiments, wherein the FXR agonist is tropifexor.

12a. The FXR agonist for use according to Embodiment 11a, wherein tropifexor is administered to said subject once daily, at a therapeutically effective dose.

13a. The FXR agonist for use according to Embodiment 11a, wherein tropifexor is administered to said subject once daily, at a dose of about 30 μg to about 250 μg, e.g. of about 60 μg to about 200 μg, e.g. of about 90 μg to about 140 μg.

14a. Tropifexor, e.g. in free form, or a salt thereof, or an amino acid conjugate thereof, for use in the treatment or in the prevention of a condition associated with mitochondrial dysfunction, e.g. a mitochondrial disease; wherein tropifexor is administered to a subject in need thereof, once daily, at a therapeutically effective dose.

15a. Tropifexor, e.g. in free form, or a salt thereof, or an amino acid conjugate thereof, for use in the treatment or prevention of a condition associated with mitochondrial dysfunction, e.g. a mitochondrial disease, wherein tropifexor is administered to a subject in need thereof, once daily, at a dose of about 30 μg to about 250 μg, e.g. of about 60 μg to about 200 μg, e.g. of about 90 μg to about 140 μg.

### Embodiments (b)

1b. A method for the treatment of a condition or a disease associated with mitochondrial dysfunction, e.g. a mitochondrial disease, in a subject in need thereof, comprising administering once daily to said subject a therapeutically effective amount of a FXR agonist.

2b. A method for the prevention of a condition or a disease associated with mitochondrial dysfunction, e.g. a mitochondrial disease, in a subject in need thereof, comprising administering once daily to said subject a therapeutically effective amount of a FXR agonist.

3b. A method for the treatment or for the prevention of a condition or a disease associated with mitochondrial dysfunction and which condition or disease is mediated by farnesoid X receptors (FXRs), in a subject in need thereof, comprising administering to said subject once daily, a therapeutically effective amount of a FXR agonist.

4b. The method according to any one of Embodiments 1b and 2b, wherein the FXR agonist is selected from tropifexor, obeticholic acid, nidufexor, cilofexor, TERN-101, EDP-305, PXL007, AGN242266 and MET409.

5b. The method according to Embodiment 4b, wherein the FXR agonist is obeticholic acid.

6b. The method according to Embodiment 5b, wherein obeticholic acid is administered at a daily dose of about 5 mg, of about 10 mg, of about 15 mg, of about 20 mg, of about 25 mg, of about 30 mg, of about 40 mg or of about 50 mg.

7b. The method according to Embodiment 7b, wherein the FXR agonist is tropifexor.

8b. The method according to Embodiment 6b, wherein tropifexor is administered at a daily dose of about 90 μg to about 250 μg, e.g. of about 140 μg to about 200 μg.

### Embodiments (c)

1c. A pharmaceutical composition comprising a FXR agonist, or a pharmaceutically acceptable salt thereof, and at least one pharmaceutically acceptable excipient, for use in the treatment of a condition or a disease associated with mitochondrial dysfunction, in a subject in need thereof, comprising a therapeutically effective amount of at least one FXR agonist, wherein the pharmaceutical composition is to be administered once daily.

2c. A pharmaceutical composition comprising an FXR agonist for use according to any of Embodiments 1a to 15a, and at least one pharmaceutically acceptable excipient.

### Embodiments (d)

1d. Use of FXR agonist as defined in any one of Embodiments 1a to 15a, or a pharmaceutically acceptable salt thereof, for the manufacture of a medicament for the treatment of a condition or a disease associated with mitochondrial dysfunction, e.g. mitochondrial disease.

2d. Use of tropifexor in the manufacture of a medicament for treating or preventing a condition or a disease associated with mitochondrial dysfunction, e.g. a mitochondrial disease, wherein tropifexor is to be administered once daily, at a dose daily dose, of about 90 μg to about 250 μg, about 140 μg to about 200 μg, and wherein tropifexor is administered in the evening.

### Embodiments (e)

1e. Use of a pharmaceutical composition comprising an FXR agonist according to any one of Embodiment 1a to 15a, or a pharmaceutically acceptable salt thereof, and at least one pharmaceutically acceptable excipient, for the manufacture of a medicament for the treatment of a condition mediated by Farnesoid X receptor (FXR), in particular liver disease or intestinal disease.

A FXR agonist, a method, a pharmaceutical composition, or a use, according to any one of above listed Embodiments, for treating or preventing a condition or a disease associated with mitochondrial dysfunction, e.g. a mitochondrial disease.

A FXR agonist, a method, a pharmaceutical composition, or a use, according to any one of above listed Embodiments, wherein the condition or a disease associated with mitochondrial dysfunction, is selected from neurodegenerative diseases; cardiovascular diseases; diabetes and metabolic syndrome; autoimmune diseases; neurobehavioral and psychiatric diseases; gastrointestinal disorders; fatiguing illnesses; musculoskeletal diseases; cancer; chronic infections; and kidney injury and diseases.

Tropifexor is administered at a dose (e.g. daily dose) of about 30 μg to about 250 μg, e.g. of about 60 μg to about 200 μg.

Obeticholic acid is administered at a daily dose of about 5 mg, of about 10 mg, of about 15 mg, of about 20 mg, of about 25 mg, of about 30 mg, of about 40 mg or of about 50 mg.

In certain embodiments, disclosed herein are methods of treating or preventing the adverse effects of administration of compounds which exhibit mitochondrial toxicity comprising the administration of a therapeutically effective amount of a compound as disclosed herein to a subject in need thereof. The adverse effect is selected from the group consisting of abnormal mitochondrial respiration, abnormal oxygen consumption, abnormal extracellular acidification rate, abnormal mitochondrial number, abnormal lactate accumulation, and abnormal ATP levels.

In yet another aspect, a pharmaceutical unit dosage form composition comprising about 90 μg, about 140 μg, about 150 μg, about 160 μg, about 170 μg, about 180 μg, about 190 μg, about 200 μg, about 210 μg, about 220 μg, about 230 μg, about 240 μg or about 250 μg of tropifexor suitable for oral administration once daily. Such unit dosage form compositions may be in a form selected from a liquid, a tablet, a capsule.

### Definitions

For purposes of interpreting this specification, the following definitions will apply and whenever appropriate, terms used in the singular will also include the plural and vice versa.

As used herein, the term “about” in relation to a numerical value x means+/−10%, unless the context dictates otherwise.

As used herein, a “FXR agonist”/“FXR agonists” refer to any agent that is capable of binding and activating farnesoid X receptor (FXR) which may be referred to as bile acid receptor (BAR) or NR1H4 (nuclear receptor subfamily 1, group H, member 4) receptor. FXR agonist may act as agonists or partial agonists of FXR. The agent may be e.g. a small molecule, an antibody or a protein, preferably a small molecule. The activity of a FXR agonist may be measured by several different methods, e.g. in an in vitro assay using the fluorescence resonance energy transfer (FRET) cell free assay as described in Pellicciari, et al. Journal of Medicinal Chemistry, 2002 vol. 15, No. 45:3569-72.

The FXR agonist as used herein refers, for example, to compounds disclosed in: WO2016/096116, WO2016/127924, WO2017/218337, WO2018/024224, WO2018/075207, WO2018/133730, WO2018/190643, WO2018/214959, WO2016/096115, WO2017/118294, WO2017/218397, WO2018/059314, WO2018/085148, WO2019/007418, CN109053751, CN104513213, WO2017/128896, WO2017/189652, WO2017/189663, WO2017/189651, WO2017/201150, WO2017/201152, WO2017/201155, WO2018/067704, WO2018/081285, WO2018/039384, WO2015/138986, WO2017/078928, WO2016/081918, WO2016/103037, WO2017/143134.

The FXR agonist is preferably selected from: tropifexor, nidufexor, obeticholic acid (6a-ethyl-chenodeoxycholic acid), cilofexor (GS-9674, Px-102),

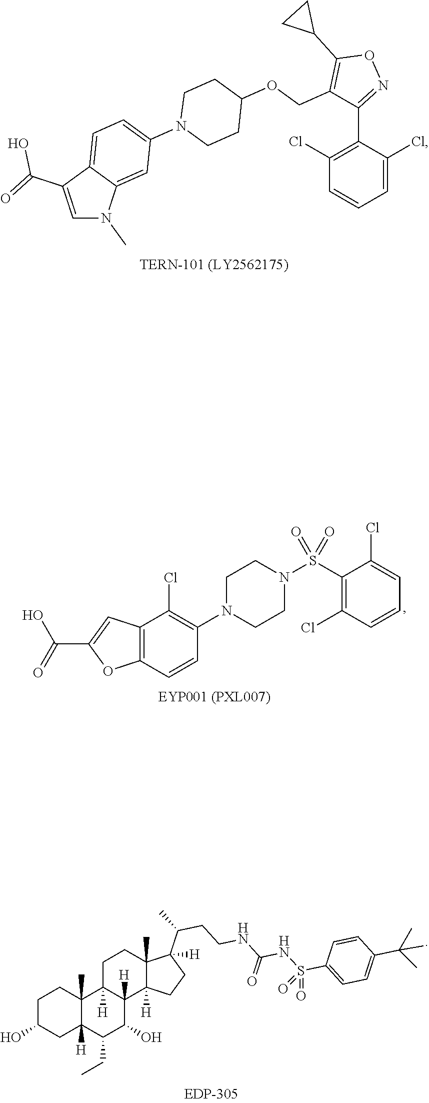

As used herein, the terms “salt” or “salts” refer to an acid addition or base addition salt of a compound of the invention. “Salts” include in particular “pharmaceutical acceptable salts”, and both can be used interchangeably herein.

As used herein, the term “pharmaceutically acceptable” means a nontoxic material that does not substantially interfere with the effectiveness of the biological activity of the active ingredient(s).

As used herein the term “prodrug” refers to a compound that is converted in vivo to the compounds of the present invention. A prodrug is active or inactive. It is modified chemically through in vivo physiological action, such as hydrolysis, metabolism and the like, into a compound of this invention following administration of the prodrug to a subject. The suitability and techniques involved in making and using pro-drugs are well known by those skilled in the art. Suitable prodrugs are often pharmaceutically acceptable ester derivatives.

As used herein, the terms “subject” or “subjects” refer to a mammalian organism, preferably a human being, who is diseased with the condition (i.e. disease or disorder) of interest and who would benefit from the treatment, e.g. a patient.

As used herein, a subject is “in need of” a treatment if such subject would benefit biologically, medically or in quality of life from such treatment.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition, such as e.g. liver cirrhosis; or to preventing or delaying a need for liver transplantation.

As used herein, the term nonalcoholic fatty liver disease (NAFLD) may refer to nonalcoholic fatty liver (NAFL), noncirrhotic NASH, and NASH with cirrhosis.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

As used herein, the term “therapeutically effective amount” refers to an amount of the compound, which is sufficient to achieve the stated effect. Accordingly, a therapeutically effective amount used for the treatment or prevention of a liver disease or disorder as hereinabove defined is an amount sufficient for the treatment or prevention of such a disease or disorder.

By “therapeutic regimen” is meant the pattern of treatment of an illness, e.g., the pattern of dosing used during the treatment of the disease or disorder.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis and liver fibrosis.

As used herein, the term NAFLD may encompass the different stages of the disease: hepatosteatosis, NASH, fibrosis and cirrhosis.

As used herein, the term NASH may encompass steatosis, hepatocellular ballooning and lobular inflammation.

As used herein, the term “condition or disease associated with mitochondrial dysfunction”, e.g. mitochondrial diseases, are conditions or diseases which result from failures of the mitochondria, and are diagnosed according to the mitochondrial disease diagnosis criteria.

As used herein, the term “mitochondrial hepatopathies” encompasses a plurality of disease, for example as disclosed in Sokol R J, Treem W R. Mitochondria and childhood liver diseases. J Pediatr GastroenterolNutr 1999; 28:4-16.

As herein defined, “combination” refers to either a fixed combination in one unit dosage form (e.g., capsule, tablet, or sachet), free (i.e. non-fixed) combination, or a kit of parts for the combined administration where a FXR agonist, such as tropifexor, and the one or more additional therapeutic agents may be administered independently at the same time or separately within time intervals, especially where these time intervals allow that the combination partners show a cooperative, e.g. synergistic effect.

The term “pharmaceutical combination” as used herein means a pharmaceutical composition that results from the combining (e.g. mixing) of more than one active ingredient and includes both fixed and free combinations of the active ingredients.

As used herein, the term “qd” means a once daily administration.

The term “dose” refers to a specified amount of a drug administered at one time. As used herein, the dose is the amount of the drug that elicits a therapeutic effect. The dose would, for example, be declared on a product package or in a product information leaflet. For example, for tropifexor, the term “dose” when used in relation to tropifexor is the amount of tropifexor in free form. Since tropifexor can be present in the form of a salt or of an amino acid conjugate, the amount of the respective salt former (e.g. the respective acid) or of the amino acid, has to be added accordingly.

### Modes of Administration

The pharmaceutical composition of the invention can be formulated to be compatible with its intended route of administration (e.g. oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration. The pharmaceutical compositions compatible with each intended route are well known in the art.

### Timing of the Administration

The FXR agonist of the invention, as herein defined in above listed embodiments, may be administered in the morning or in the evening.

In one embodiment, the term “administration in the evening” is generally defined as administration any time from about 6 μm to about 12 pm, e.g. from about 8 pm to about 11 pm, preferably around 9 pm. Administration in the evening may be before the evening meal, with the evening meal or after the evening meal.

In one embodiment, the term “administration in the evening” refers to administration shortly before or at bedtime. In one embodiment, the term “administration in the evening” refers to administration shortly before bedtime. In one embodiment, the term “administration in the evening” refers to administration at bedtime. Unless otherwise specified herein, the term “bedtime” has the normal meaning of a time when a person retires for the primary sleep period during a twenty-four hour period of time. The administration shortly before bedtime means that the FXR agonist as herein defined, is administered within about 1-2 hours prior to a person's normal rest or sleep (typically 4 to 10-hours) period.

### Diseases

Mitochondrial dysfunction, characterized by a loss of efficiency in the electron transport chain and reductions in the synthesis of high-energy molecules, such as adenosine-5′-triphosphate (ATP), is a characteristic of aging, and chronic diseases including neurodegenerative diseases, such as Alzheimer's disease, Parkinson's disease, Huntington's disease, amyotrophic lateral sclerosis, and Friedreich's ataxia; cardiovascular diseases, such as atherosclerosis and other heart and vascular conditions; diabetes and metabolic syndrome; autoimmune diseases, such as multiple sclerosis, systemic lupus erythematosus, and type 1 diabetes; neurobehavioral and psychiatric diseases, such as autism spectrum disorders, schizophrenia, and bipolar and mood disorders; gastrointestinal disorders; fatiguing illnesses, such as chronic fatigue syndrome and Gulf War illnesses; musculoskeletal diseases, such as fibromyalgia and skeletal muscle hypertrophy/atrophy; cancer; and chronic infections. (Nicolson, Integr. Med. 13:35-43 (2014); Sorrentino et al., Annual Review of Pharmacology and Toxicology 2018, 58:1, 353-389).

In one embodiment, the condition or disease or disorder associated with mitochondrial dysfunction, is a gastrointestinal disease or disorder such as idiopathic inflammatory bowel disease, e.g. Crohn's disease and ulcerative colitis.

In another embodiment, the condition or disease or disorder associated with mitochondrial dysfunction, is a liver disease or disorder, e.g. as defined herein, or renal fibrosis. Oxidative stress plays an important role in the pathogenesis of renal fibrosis, by causing damage to mitochondria, and subsequently inducing renal injury. Qin et al., Chin. Med. J. 2018; 131(22): 2769-2772).

In yet another embodiment, the condition or disease or disorder associated with mitochondrial dysfunction, is a kidney disease, e.g., kidney injury such as acute and chronic kidney injury; and diabetic kidney disease. (Tang et al., J Am Soc Nephrol. 2016, 27:1869-1872; Galvan et al., Kidney Int. 2017, 92(5): 1051-1057; Forbes, Nature Reviews Nephrology 14, 291-312 (2018)).

In yet another embodiment, the condition or disease or disorder associated with mitochondrial dysfunction, is a mitochondrial hepatopathy, e.g., a primary mitochondrial hepatopathy or a secondary mitochondrial hepatopathy.

### Subjects

According to the invention, the subjects receiving the FXR agonist of the invention can be affected or at risk of a conditions for which mitochondrial dysfunction is a major mechanism, e.g. as hereinabove defined.

### Dosing Regimens

Depending on the compound used, the targeted disease or disorder and the stage of such disease or disorder, the dosing regimen, i.e. administered doses and/or frequency of each component of the pharmaceutical combination may vary. The dosing frequency will depend on; inter alia, the phase of the treatment regimen.

According to the invention, tropifexor (as hereinabove defined), is administered at a dose of about 30 μg to about 250 μg, e.g. about 60 μg to about 200 μg, e.g. 90 μg to about 140 μg. Such doses may be for oral administration. Preferably, tropifexor (as hereinabove defined), is administered at a dose of about 90 μg, or about 140 μg.

Obeticholic acid is to be administered at a daily dose of about 5 mg, of about 10 mg, of about 15 mg, of about 20 mg, of about 25 mg, of about 30 mg, of about 40 mg or of about 50 mg.

In some embodiments, obeticholic acid as herein defined, is to be administered at a daily dose of about 25 mg.

### EXAMPLES

### Example 1: FXR Activation by Tropifexor (TXR) Restores Hepatic Mitochondrial Function in Dietary Mouse NASH Model

This study aims at elucidating the contribution of mitochondria dysfunction to NASH progression and investigating the molecular mechanisms underlying tropifexor-mediated protection against oxidative stress in NASH in rodents.

Methods: To induce NASH, C57BL/6J mice received a diet (high in fat, carbohydrate, cholesterol, combined with ad libitum consumption of fructose-sucrose solution) for 20 weeks followed by tropifexor treatment for the last 12 weeks. Molecular, structural and functional analysis was applied to assess oxidative stress and mitochondria function in NASH model.

Similar to human disease, transition from steatosis to NASH with fibrosis in NASH mice is associated with mitochondrial dysfunction characterized by structural and functional alterations. Decline in hepatic mitochondrial function is manifested in HF/NASH mice by decreased activities of tricarboxylic acid (TCA) cycle and electron transport chain (ETC) as well as diminished ATP production. This is accompanied by oxidative damage and depletion of antioxidant enzymes. As shown herein, tropifexor restored hepatic mitochondrial function in NASH mice. This was manifested by a marked increase in mitochondrial function with concomitant restoration of oxidative balance associated with increased antioxidant defenses and decreased oxidative stress.

### Tropifexor Decreases Oxidative Stress and Restores Antioxidant Defenses in Dietary HF/NASH Model

The pathophysiology and progression of NASH is influenced by multiple factors, among which oxidative mitochondrial dysfunction is a central feature of steatosis to NASH transition. Longitudinal assessment of hepatic oxidative stress and mitochondrial function in HF/NASH model revealed structural and functional mitochondrial alterations. A progressive decline in mitochondrial function manifested by a decreased tricarboxylic acid (TCA) cycle assessed by citrate synthase (CS) activity, decline in complex I activity of electron transport chain (ETC) and diminished ATP production in HF/NASH livers. This accompanied by oxidative damage indicated by elevated levels of malondialdehyde (MDA), a cytotoxic product of lipid peroxidation, and depletion in antioxidants activity, glutathione peroxidase (GPx) and superoxide dismutase (SOD), in HF/NASH livers. Interestingly, a transient increase in the activities of CS and GPx as well as in ATP synthesis, indicative of mitochondrial adaptation and observed at earlier time points (week 8 and 12 HF/NASH) was lost in the more advanced stages of NASH (week 16 and 20 HF/NASH). TEM on HF/NASH livers revealed a marked decrease in liver mitochondrial size in mice fed HF/NASH diet. Frequency distributions for mitochondrial size showed a significantly greater prevalence of small mitochondria (<0.5 μm2) and decreased frequency of larger mitochondria (>0.5 μm2) at week 20 HF/NASH.

Mitochondrial impairment was further underpinned through transcriptome-wide RNA profiling which showed progressive deregulation of the main processes involved in mitochondrial function. In conclusion, these data reveal that, similar to human NASH, progression to NASH with fibrosis in our experimental model is associated with oxidative mitochondrial dysfunction.

Tropifexor markedly decreased levels of oxidative stress in HF/NASH livers, as shown by the reduction in MDA and 4-H NE, products of lipid peroxidation and restoration of mitochondrial DNA (mtDNA), a natural surrogate of oxidative DNA damage. Moreover, serum levels of y-glutamyltranspeptidase (GGT), a well-established marker of systemic oxidative stress, increased in HF/NASH mice, were decreased with tropifexor treatment. Next, the activity of catalase, SOD and GPx, the three primary scavenger enzymes involved in detoxifying reactive oxygen species was evaluated in tropifexor-treated HF/NASH mice. GPx activitys in serum and liver was restored with 0.9 mg/kg tropifexor. Liver SOD activity was fully restored by 0.3 mg/kg TXR and increased above the levels observed in ND mice at 0.9 mg/kg.

Transcriptome analysis confirmed regulation of pathways involved in oxidative stress and mitochondrial dysfunction in TXR-treated HF/NASH mice. Specifically, Tropifexor induced antioxidant gene expression belonging to GPx and Glutathione-S-transferase (GST) superfamilies and involved in glutathione-dependent detoxification. Tropifexor either restored expression of investigated genes to ND levels (Gpx3, Gpx8), or markedly increased their expression beyond the ND levels (Gpx2, Gst family). Interestingly, prostaglandin-D synthase (Pdgs), belonging to sigma class GST, was one of the genes highest elevated by TXR in HF/NASH livers. Together, these data highlight a global effect of tropifexor on regulation of transcriptional activity of glutathione-dependent detoxifying pathways. Oxidative stress can regulate the sensitivity of hepatocytes to cell death pathways. Apoptosis in liver was increased in mice fed a HF/NASH diet for 20 weeks and tropifexor attenuated cell death in a dose dependent manner, as measured by a decrease in cleaved-caspase 3 staining and phospho-p38 protein levels. Moreover, HF/NASH-induced expression of pro-apoptotic pathways decreased, while the expression of anti-apoptotic genes increased in TXR-treated groups (heat map-RNAseq).

### Tropifexor Restores Mitochondrial Function in HF/NASH Model

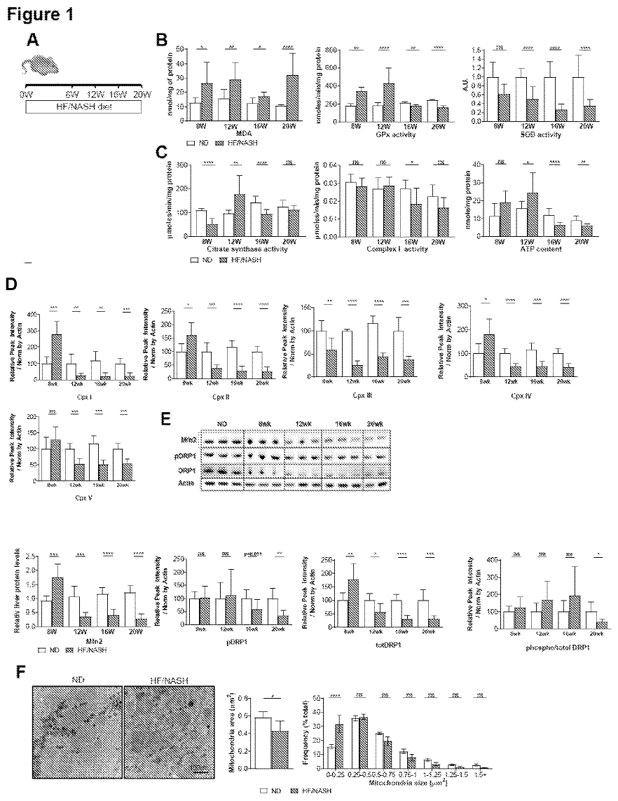

Next, we evaluated if tropifexor-mediated decrease in oxidative stress and restoration of antioxidant defenses translated into improved hepatic mitochondria function in HF/NASH livers. Tropifexor dose-dependently restored citrate synthase (CS) activity (FIG. 1). Moreover, FXR treatment counteracted oxidative phosphorylation dysfunction by restoring Complex I and Complex II activities as well as improving ATP synthesis in HF/NASH livers. Consistent with impairment of mitochondrial respiratory chain function, protein levels of all five complexes constituting the oxidative phosphorylation (OXPHOS) chain were significantly decreased in HF/NASH livers (FIG. 1). Tropifexor increased the content of all five respiratory chain proteins in HF/NASH livers with full restoration observed already at 0.1 mg/kg. Tropifexor treatment in HF/NASH mice did not lead to changes in expression of genes involved in mitochondrial biogenesis including PGC-1a, Nrf1 or Tfam thus, excluding a role of FXR in regulation of mitochondrial biogenesis. Collectively, this data provide strong evidence that TXR restores hepatic mitochondrial dysfunction in NASH mice by inducing respiratory chain function, TCA cycle and improving ATP production.

### FXR Treatment Promotes Healthy Mitochondrial Function in Mice

Tropifexor-mediated restoration of mitochondrial function in NASH livers can be an indirect consequence of ameliorated NASH, or direct effect as hinted by stimulation of TCA, ETC and ATP synthesis above the levels observed by normal diet-fed mice.

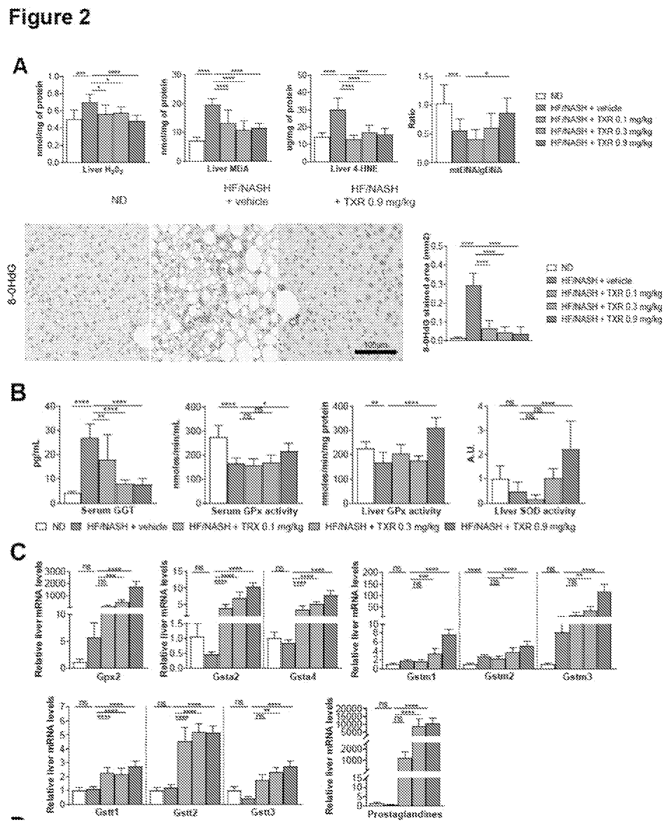

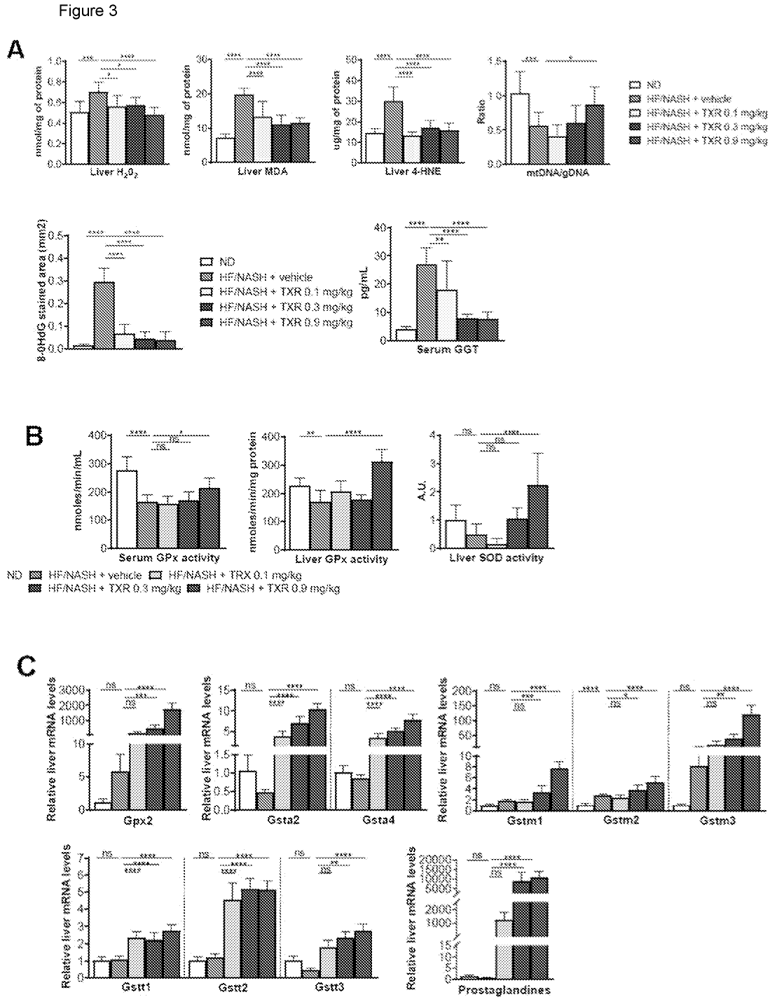

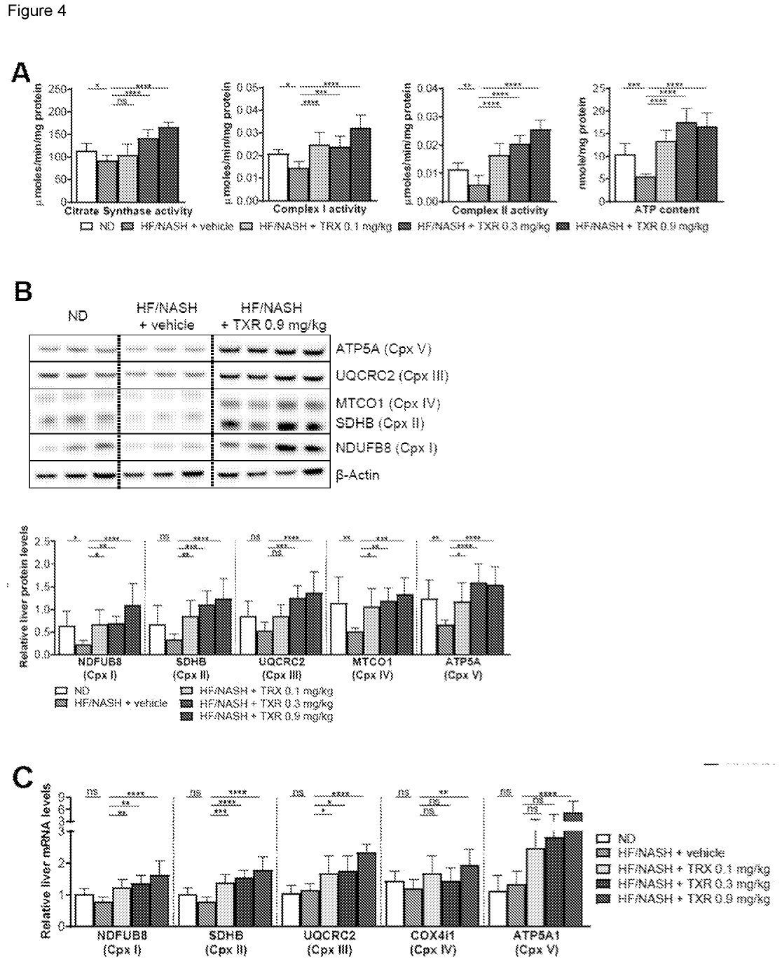

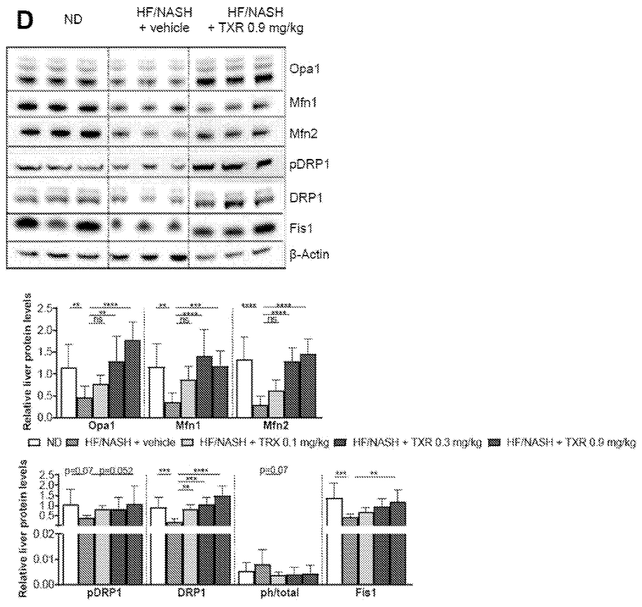

In order to confirm that tropifexor directly regulates mitochondrial function, wild-type mice fed a normal diet were treated with 0.9 mg/kg tropifexor for 4 weeks (FIG. 2). Tropifexor treatment resulted in robust FXR target gene induction (SHP, BSEP, FGF15) or repression (CYP8B1) in the liver and ileum, and increased serum FGF15 levels (FIG. 2).

Tropifexor significantly improved mitochondrial function in wild-type livers as demonstrated by increased activities of respiratory chain complexes I and II, and citrate synthase (FIG. 2). Moreover, ATP content markedly increased following tropifexor treatment (FIG. 2). OXPHOS protein analysis did not reveal significant changes in the content of mitochondrial respiratory chain units except for Complex V (ATP synthase), which was significantly increased in tropifexor-treated wild type livers (FIG. 2). While GPx activity was not alter, the catalase activity significantly increased in tropifexor-treated wild-type livers (FIG. 2), constant with the reports that under physiological conditions, catalase-dependent reactions drive the ROS elimination process 37. The analysis of GPx and GST expression revealed that the hepatic transcript levels increased in X- and X-fold, respectively in tropifexor compared with wild-type controls. Furthermore, RNA Seq data on redox pathways data provide an evidence for tropifexor promoting healthy mitochondrial function in mice.

This study provides first evidence that tropifexor, potent and selective FXR agonist in Phase IIb development for NASH, regulates hepatic mitochondrial function in diet-induced mouse model of NASH. As shown herein, tropifexor repairs hepatic mitochondrial dysfunction by combating oxidative stress and restoring antioxidant defenses in the setting of mitochondrial dysfunction in NASH.

### Example 2: Role of Tropifexor in the Reductions of Hepatic Fat and Serum Alanine Aminotransferase in Patients with Fibrotic NASH after 12 Weeks of Therapy (FLIGHT-FXR Part C Interim Results)

Parts A and B of study CLJN452A2202 in NASH patients have investigated tropifexor at doses ranging from 10 to 90 μg daily for 12 weeks. Tropifexor exhibited a clear dose response for target engagement (FGF19) and biologic activity (GGT). ALT and hepatic fat fraction were reduced across all tropifexor doses (10, 30, 60 and 90 μg) compared to placebo. The study showed that Tropifexor was generally well tolerated up to 90 μg daily without safety signals. Results from the first two parts (A and B, study CLJN452A2202) demonstrated anti-inflammatory and anti-steatotic efficacy of 60 and 90 μg of tropifexor based on biomarkers, and favorable safety at Week 12.

FLIGHT-FXR (NCT02855164) is a phase 2 randomized, double blind, placebo-controlled, 3-part, adaptive-design study to assess the safety, tolerability, and efficacy of several doses of tropifexor (LJN452) in patients with non-alcoholic steatohepatitis (NASH).

METHODS: In Part C, the effects of higher doses of tropifexor on biomarkers and histology will be evaluated over 48 weeks in patients with biopsy-proven NASH and fibrosis stages 2-3. In all, 152 patients (64% females) were randomized to receive placebo (N=51), tropifexor 140 μg (N=50) or tropifexor 200 μg (N=51) once daily. Prespecified endpoints assessed at week 12 included overall safety and changes in alanine aminotransferase (ALT), hepatic fat fraction (HFF), gamma glutamyl transferase (GGT), and body weight.

RESULTS: Prespecified endpoints were met for tropifexor at a dose of 200 μg. Efficacy results are presented in Table 2.

TABLE 2
Least squares means of absolute changes in ALT, GGT, and body weight, and relative
change in HFF from baseline to Week 12 estimated in repeated measures or analysis of
covariance models (full analysis set)

| | | Tropifexor | Tropifexor |
|---|---|---|---|
| Biomarkers | Placebo (N = 51) | 140 μg (N = 50) | 200 μg (N = 51) |
| ALT (U/L) | −8.9 (4.19) | −20.1 (4.57) | −23.6 (4.48) |
| | n = 49 | n = 41; P = 0.058 | n = 39; P = 0.013 |
| Relative change | −10.26 (4.21) | −16.99 (4.64) | −31.37 (4.30) |
| in HFF* (%) | n = 51 | n = 49; P = 0.209 | n = 51; P<0.001 |
| GGT (U/L) | −2.5 (3.55) | −39.2 (3.70) | −40.9 (3.62) |
| | n = 49 | n = 44; P<0.001 | n = 46; P<0.001 |
| Body weight (kg) | −1.14 (0.36) | −2.46 (0.38) | −3.20 (0.37) |
| | n = 50 | n = 46; P = 0.010 | n = 46; P<0.001 |

*Measured as magnetic resonance imaging-proton density fat fraction (MRI-PDFF). Data are presented as LS mean change (SE) with 2-sided P values reported for statistical significance ALT, alanine aminotransferase; GGT, gamma glutamyl transferase; HFF, hepatic fat fraction; LS, least square; SE, standard error;

Relative HFF reduction (without imputation for missing values) by ≥30% was achieved in 20%, 32%, and 64% of patients in the placebo, Tropifexor 140 μg, and Tropifexor 200 μg groups, respectively. The frequency of serious adverse events was low and comparable across groups. Among patients with pruritus, >60% in both Tropifexor groups and all in the placebo group experienced events with mild (Grade 1) severity. Treatment discontinuation rates due to pruritus were low (Tropifexor 140 μg: n=1 [2%]; Tropifexor 200 μg: n=3 [6%]; placebo: 0%). A dose-related increase in low density lipoprotein-cholesterol (LDL-C) was seen. None of the lipid changes led to treatment discontinuation or dose reduction.

In this prespecified interim analysis of Part C, higher doses of Tropifexor resulted in robust and dose-dependent decreases in ALT, HFF, GGT and body weight with good safety and tolerability after 12 weeks of treatment. Similar to other FXR agonists, these higher doses were associated with mild pruritus and minor dose-related increase in LDL-C.

### Example 3: Study to Evaluate Safety and Efficacy of FXR Agonist for Treating Mitochondrial Disease in Subject in Need Thereof

Subjects with suspected or confirmed mitochondrial disease, e.g. mitochondrial hepatopathy will be enrolled. Prior to treatment, subjects will undergo a Screening Visit. If eligible, each participant will return for the Day 1 study visit and begin dosing with the investigational drug, e.g. FXR agonist as described herein.

The primary outcome measure is the functional assessment of the patient's clinical outcomes, e.g. by International Paediatric Mitochondrial Disease Score (IPMDS) or other recognized mitochondrial disease score metrics. Secondary outcome measures included the measurement of biochemical and radiological parameters. Furthermore, tolerability and quality of life of the subjects will be determined.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1-9. (canceled)

10. A method for treating a condition or a disease associated with mitochondrial dysfunction, wherein said condition or disease is mediated by a farnesoid X receptor (FXR), in a subject in need thereof, comprising administering to a subject in need thereof a therapeutically effective amount of a FXR agonist once daily.

11. The method according to claim 10, wherein the FXR agonist is selected from tropifexor, obeticholic acid, nidufexor, cilofexor, TERN-101, EDP-305, PXL007, AGN242266 and MET409.

12. The method according to claim 10, wherein the FXR agonist is obeticholic acid.

13. The method according to claim 12, wherein obeticholic acid is administered at a daily dose of about 5 mg, of about 10 mg, of about 15 mg, of about 20 mg, of about 25 mg, of about 30 mg, of about 40 mg or of about 50 mg.

14. The method according to claim 10, wherein the FXR agonist is tropifexor.

15. The method according to claim 14, wherein tropifexor is administered at a daily dose of about 30 μg to about 250 μg.

16. The method according to claim 14, wherein tropifexor is administered at a daily dose of about 60 μg to about 200 μg.

17. The method according to claim 10, wherein the FXR agonist is administered in the evening.
